SEQUENCE LISTING (43 sequences)

SEQ ID NO: 1 - DNA, 1603 bp, Human
  CDS (38)..(577)
cgaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtgggctacagagatgaaagaggggtgggatccacactgagagagtggagagtgacatgtgctggacactgtccatgaagcactgagcagaaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgg

SEQ ID NO: 2 - protein, 180 aa, Human
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEECTPGPDGAAGSPDAWV

SEQ ID NO: 3 - DNA, 492 bp, Human
  CDS (1)..(489)
gcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctga

SEQ ID NO: 4 - protein, 163 aa, Human
APLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEECTPGPDGAAGSPDAWV

SEQ ID NO: 5 - DNA, 471 bp, Human
  CDS (1)..(468)
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctga

SEQ ID NO: 6 - protein, 156 aa, Human
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEECTPGPDGAAGSPDAWV

SEQ ID NO: 7 - DNA, 708 bp, Human
  misc_feature (38)..(577): CDS
ccaaggttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctc

SEQ ID NO: 8 - protein, 16 aa, Human
CTPGPDGAAGSPDAWV

SEQ ID NO: 9 - DNA, 1541 bp, Human
  CDS (17)..(685)
ggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtcaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagctggacttacagaaataaagagctatcatgctgtggttaaaaaaaaaaaaaaaaaaaaaaaaaaaa

SEQ ID NO: 10 - protein, 223 aa, Human
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKG

SEQ ID NO: 11 - DNA, 621 bp, Human
  CDS (1)..(618)
gtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtga

SEQ ID NO: 12 - protein, 206 aa, Human
VPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKG

SEQ ID NO: 13 - DNA, 600 bp, Human
  CDS (1)..(597)
attgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtga

SEQ ID NO: 14 - protein, 199 aa, Human
IVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKG

SEQ ID NO: 15 - protein, 13 aa, Human
VSHPYSQHLEGKG

SEQ ID NO: 16 - DNA, 27 bp, Artificial Sequence
  PSA wild-type forward primer of Table 1
atcgaattcgcacccggagagctgtgt

SEQ ID NO: 17 - DNA, 22 bp, Artificial Sequence
  PSA wild-type reverse primer of Table 1
ctgagggtgaacttgcgcacac

SEQ ID NO: 18 - DNA, 27 bp, Artificial Sequence
  PSA RP2 transcripts 1 and 2 forward primer of Table 1
atcgaattcgcacccggagagctgtgt

SEQ ID NO: 19 - DNA, 21 bp, Artificial Sequence
  PSA RP2 transcripts 1 and 2 reverse primer of Table 1
caaggcaaaaaacactgttgt

SEQ ID NO: 20 - DNA, 21 bp, Artificial Sequence
  PSA 525 forward primer of Table 1
ggtatttcaggtcagccacag

SEQ ID NO: 21 - DNA, 21 bp, Artificial Sequence
  PSA 525 reverse primer of Table 1
taaaagcagggagagagtgag

SEQ ID NO: 22 - DNA, 21 bp, Artificial Sequence
  PSA (Schulz) forward primer of Table 1
ctgggcgctgtcttgtgtctc

SEQ ID NO: 23 - DNA, 27 bp, Artificial Sequence
  PSA (Schulz) reverse primer of Table 1
atgtctagagttgataggggtgctcag

SEQ ID NO: 24 - DNA, 22 bp, Artificial Sequence
  KLK2 wild type forward primer of Table 1
gcctaaagaagaatagccaggt

SEQ ID NO: 25 - DNA, 21 bp, Artificial Sequence
  KLK2 wild type reverse primer of Table 1
tctaaggctttgatgcttcag

SEQ ID NO: 26 - DNA, 21 bp, Artificial Sequence
  KLK2 10A forward primer of Table 1
cacttgtggggtgagtcatcc

SEQ ID NO: 27 - DNA, 27 bp, Artificial Sequence
  KLK2 10A reverse primer of Table 1
atgtctagatgggacaggggcactcag

SEQ ID NO: 28 - DNA, 21 bp, Artificial Sequence
  b2-microgloblin forward primer of Table 1
tgaattgctatgtgtctgggt

SEQ ID NO: 29 - DNA, 21 bp, Artificial Sequence
  b2-microgloblin reverse primer of Table 1
cctccatgatgctgcttacat

SEQ ID NO: 30 - DNA, 21 bp, Artificial Sequence
  PSA wild-type forward primer of Table 2
gcatcaggaacaaaagcgtga

SEQ ID NO: 31 - DNA, 21 bp, Artificial Sequence
  PSA wild-type reverse primer of Table 2
cctgaggaatcgattcttcag

SEQ ID NO: 32 - DNA, 21 bp, Artificial Sequence
  PSA RP2 transcripts 1 and 2 forward primer of Table 2
gagtgtacgcctgggccagat

SEQ ID NO: 33 - DNA, 22 bp, Artificial Sequence
  PSA RP2 transcripts 1 and 2 reverse primer of Table 2
ctgagggtgaacttgcgcacac

SEQ ID NO: 34 - DNA, 21 bp, Artificial Sequence
  PSA 525 forward primer of Table 2
ctcactctctccctgctttta

SEQ ID NO: 35 - DNA, 27 bp, Artificial Sequence
  PSA 525 reverse primer of Table 2
atgtctagagttgataggggtgctcag

SEQ ID NO: 36 - DNA, 21 bp, Artificial Sequence
  PSA (Schulz) forward primer of Table 2
ctgggcgctgtcttgtgtctc

SEQ ID NO: 37 - DNA, 21 bp, Artificial Sequence
  PSA (Schulz) reverse primer of Table 2
caacgagaggccacaagcacc

SEQ ID NO: 38 - DNA, 22 bp, Artificial Sequence
  KLK2 wild type forward primer of Table 2
gcctaaagaagaatagccaggt

SEQ ID NO: 39 - DNA, 21 bp, Artificial Sequence
  KLK2 wild type reverse primer of Table 2
tctaaggctttgatgcttcag

SEQ ID NO: 40 - DNA, 21 bp, Artificial Sequence
  KLK2 10A forward primer of Table 2
cacttgtggggtgagtcatcc

SEQ ID NO: 41 - DNA, 27 bp, Artificial Sequence
  KLK2 10A reverse primer of Table 2
atgtctagatgggacaggggcactcag

SEQ ID NO: 42 - DNA, 21 bp, Artificial Sequence
  b2-microgloblin forward primer of Table 2
tgaattgctatgtgtctgggt

SEQ ID NO: 43 - DNA, 21 bp, Artificial Sequence
  b2-microgloblin reverse primer of Table 2
cctccatgatgctgcttacat

(19) United States
(12) Patent Application Publication
(10) Pub. No.: US 2005/0287610 A1
(43) Pub. Date: Dec. 29, 2005

(54) Method for detecting the presence or risk of prostate cancer by detecting products of psa or klk2

(75) Inventor: Judith Ann Clements, Clayfield (AU)

Correspondence Address: HELLER EHRMAN WHITE & MCAULIFFE LLP, 1717 RHODE ISLAND AVE, NW, WASHINGTON, DC, 20036-3001, US

(73) Assignee: Qeensland University of Technology, Brisbane (AU)

(21) Appl. No.: 10/511,988

(22) PCT Filed: Apr. 16, 2003
(86) PCT No.: PCT/AU03/00454

§ 371 Date: Apr. 25, 2005

(30) Foreign Application Priority Data
Apr. 22, 2002 (AU) PS 1870

(51) Int. Cl. C12Q 1/68; G01N 33/574

(52) U.S. Cl. 435/7.23; 435/6

(57) ABSTRACT

The present invention discloses a method for detecting the presence or diagnosing the risk of cancer, especially prostate cancer, either before or after the onset of clinical symptoms, by detecting a level or functional activity of an aberrant expression product of a gene selected from PSA or KLK2, which correlates with the presence or risk of the cancer. The invention further encompasses the use of agents that modulate the level or functional activity of such an aberrant expression product for treating or preventing cancer, especially prostate cancer.

### FIELD OF THE INVENTION

THIS INVENTION relates generally to differentially expressed molecules. More particularly, the present invention relates to aberrant expression products of the PSA and KLK2 genes, which are differentially expressed, and which could be used, therefore, to discriminate between cancer and non-cancer cells and more particularly between prostate cancer and benign prostatic hyperplasia (BPH). Even more particularly, the present invention relates to a method for detecting the presence or diagnosing the risk of cancer, especially prostate cancer, either before or after the onset of clinical symptoms, by detecting a level or functional activity of an aberrant expression product of a gene selected from PSA or KLK2, which correlates with the presence or risk of the cancer. Assessing the level or functional activity of the aberrant expression product is useful as a prognostic indicator of disease outcome. The invention further encompasses the use of agents that modulate the level or functional activity of those expression products for treating or preventing cancer, especially prostate cancer.

### BACKGROUND OF THE INVENTION

Prostate cancer is the most commonly diagnosed cancer in the USA and other Western societies, including Australia (2000, Cancer Statistics 2000. CA. J American Cancer Society 50: 7-33). If organ-confined at diagnosis, the prognosis is usually excellent. However, because of a predisposition for metastasising to bone, the terminal phases of the disease may be extremely painful (Frydenburg et al., 1997, Lancet 349: 1681). So there is a critical need for earlier detection and early intervention and better indicators of clinically significant (i.e., aggressive) disease and more appropriate therapeutic interventions.

Prostate specific antigen (PSA), also known as kallikrein 3, or K3, a product of the KLK3 gene, is currently the only useful serum marker available for the diagnosis and monitoring of prostate cancer. The widespread acceptance of PSA by clinicians and recognition that elevated levels in serum often precede overt evidence of prostatic disease, has permitted earlier identification of patients with prostate cancer. However, PSA is not a cancer-specific marker as levels can also be elevated in benign prostatic disease (Frydenburg et al., 1997, supra).

The PSA-related gene, kallikrein 2, or KLK2, has also emerged as a potential marker for benign and malignant differentiation. A number of variant mRNA transcripts of PSA (PSA wild type, PSA RP2 transcript 1, PSA RP2 transcript 2, PSA 525, PSA Schulz) and KLK2 (KLK2 wild type, KLK2 10A) have also been identified in prostatic tissue, but have not been fully characterised. PSA RP2 transcript 1, PSA RP2 transcript 2, PSA 525 and KLK2 10A all include alternatively spliced intronic insertions, whilst the PSA Schulz variant begins in intron 1.

In work leading up to the present invention, the inventors sought to investigate whether the above mRNA transcripts are differentially expressed in benign prostatic hyperplasia (BPH) or malignant prostate tissue and found that the six transcripts were present in some, but not all, BPH and cancer samples, indicating that they are not specific to either BPH or cancer. However, quantitative analysis of these transcripts unexpectedly revealed that the variants, PSA RP2 transcript 2, KLK2 10A and possibly PSA RP2 transcript 1, are differentially expressed and that levels of these transcripts are significantly increased in cancer tissues when compared to those of BPH origin. It is proposed, therefore, that the expression level of the variants as well as the level or functional activity of their polypeptide products are useful as discriminating biomarkers for cancer.

### SUMMARY OF THE INVENTION

Accordingly, in one aspect of the present invention, there is provided a method for detecting the presence or diagnosing the risk of prostate cancer in a patient, comprising detecting in a biological sample obtained from the patient a level or functional activity of an aberrant expression product of a gene selected from PSA or KLK2, which correlates with the presence or risk of prostate cancer. In accordance with the present invention, this level or functional activity is different to, and preferably higher than, a reference level or functional activity of the expression product that correlates with the presence or risk of BPH. The PSA expression product is preferably selected from PSA RP2 transcript 1, PSA RP2 transcript 2, or a polypeptide product of these and is more preferably selected from PSA RP2 transcript 2 or a polypeptide product thereof. Suitably, the KLK2 expression product is selected from KLK2 10A variant transcript or a polypeptide product thereof.

It will be appreciated from the foregoing that discrimination between prostate cancer and BPH is facilitated by qualitatively or quantitatively determining the level or functional activity of an aberrant transcript or aberrant polypeptide as defined above. Such determinations may be achieved by direct or indirect techniques as known in the art. For example, one embodiment of the method comprises quantifying an aberrant polypeptide product, which may include:
contacting the biological sample with an antigen-binding molecule that is immuno-interactive with an aberrant polypeptide expressed from the gene;
measuring the concentration of a complex comprising the aberrant polypeptide and the antigen binding molecule in the contacted sample; and
relating the measured complex concentration to the concentration of the aberrant polypeptide in the sample.

Preferably, the concentration of the aberrant polypeptide in the biological sample is compared to a reference level of the aberrant polypeptide which correlates with the presence or risk of BPH.

In another embodiment, the method comprises:
measuring the level of an aberrant transcript expressed from the gene in the biological sample.

Preferably, the level of the transcript in the biological sample is compared to a reference level of the transcript polypeptide which correlates with the presence or risk of BPH.

The level of the transcript is preferably measured using a probe that comprises a nucleotide sequence which corresponds or is complementary to at least a portion of the aberrant transcript.

In yet another embodiment, the method comprises indirect analysis of the level of an aberrant polypeptide by qualitatively or quantitatively determining in the biological sample the level of an antigen-binding molecule (e.g., antibody) that is immuno-interactive with the aberrant polypeptide. In this embodiment, the method preferably comprises:
contacting the biological sample with an antigen corresponding to at least a portion of an aberrant polypeptide encoded by the gene;
measuring the concentration of a complex comprising the antigen and an antigen-binding molecule in the contacted sample; and
relating the measured complex concentration to the concentration of antigen-binding molecule in the sample to thereby determine the amount or level of the aberrant polypeptide in the sample.

In another aspect, the invention contemplates a method for detecting the presence or diagnosing the risk of prostate cancer in a patient, comprising detecting in a biological sample obtained from the patient a level or functional activity of an aberrant expression product of a gene selected from PSA or KLK2, which is higher than a reference level or functional activity of the expression product polypeptide which correlates with the presence or risk of benign prostatic hyperplasia (BPH).

In yet another aspect, the invention encompasses a method for detecting the presence or diagnosing the risk of prostate cancer in a patient, comprising detecting in a biological sample obtained from the patient a level or functional activity of an aberrant expression product selected from PSA RP2 transcript 1, PSA RP2 transcript 2, KLK2 10A transcript or a polypeptide encoded thereby, which correlates with the presence or risk of prostate cancer.

In still another aspect, the invention contemplates a method for detecting the presence or diagnosing the risk of prostate cancer in a patient, comprising detecting in a biological sample obtained from the patient a level or functional activity of an aberrant expression product selected from PSA RP2 transcript 2, KLK2 10A transcript or a polypeptide encoded thereby, which correlates with the presence or risk of prostate cancer.

In even yet another aspect, the invention provides a method for detecting the presence or diagnosing the risk of prostate cancer in a patient, comprising detecting in a biological sample obtained from the patient a level or functional activity of a transcript selected from PSA RP2 transcript 1, PSA RP2 transcript 2 or KLK2 10A, which correlates with the presence or risk of prostate cancer.

In still another aspect, the invention contemplates a method for detecting the presence or diagnosing the risk of prostate cancer in a patient, comprising detecting in a biological sample obtained from the patient a level or functional activity of a transcript selected from PSA RP2 transcript 2 or KLK2 10A, which correlates with the presence or risk of prostate cancer.

In a further aspect, the invention contemplates a method for detecting the presence or diagnosing the risk of metastatic prostate cancer in a patient, comprising detecting in a biological sample obtained from the patient a level or functional activity of an aberrant expression product of a gene selected from PSA and KLK2, which correlates with the presence or risk of prostate cancer, wherein the biological sample comprises a fluid or tissue other than prostate tissue. The expression product may be present intracellularly or in soluble form. Suitably, the biological sample comprises a biological fluid, which is preferably selected from seminal fluid, whole blood, serum or lymphatic fluid. The metastatic prostate cancer is preferably associated with metastasis to a bone or lymph node of the patient.

In still a further aspect, the invention provides a method for detecting the presence or diagnosing the risk of organ-confined prostate cancer in a patient, comprising detecting in a biological sample obtained from the patient the absence of a level or functional activity of an aberrant expression product of a gene selected from PSA and KLK2, which correlates with the presence or risk of prostate cancer, wherein the biological sample comprises a fluid or tissue other than prostate tissue.

In preferred embodiments of the above methods, the level of an aberrant transcript is quantified using a nucleic acid amplification technique that quantifies the aberrant transcript in real-time.

In another aspect, the invention encompasses the use of at least a portion of an aberrant expression product as broadly described above, or the use of a probe comprising a nucleotide sequence which corresponds or is complementary to at least a portion of an aberrant transcript as broadly described above, or the use of one or more antigen-binding molecules that are immuno-interactive specifically with an aberrant polypeptide as broadly described above, in the manufacture of a kit for qualitatively of quantitatively determining a level or functional activity of an aberrant expression product of a gene selected from PSA or KLK2, which correlates with the presence or risk of prostate cancer.

In still yet another aspect, the invention contemplates the use of an agent in the manufacture of a medicament for treating and/or preventing prostate cancer, wherein the agent is optionally formulated with a pharmaceutically acceptable carrier and is identifiable by a screening assay comprising:
contacting a preparation comprising at least a portion of an aberrant expression product of a gene selected from PSA or KLK2, or a variant or derivative of the expression product, with the agent; and
detecting a change in the level or functional activity of the at least a portion of the expression product(s) or the variant or derivative.

In one embodiment of this type, the agent is an antisense oligonucleotide or ribozyme that binds to, or otherwise interacts specifically with, an aberrant transcript. In another embodiment of this type, the agent is selected from one or more antigen-binding molecules that are immuno-interactive with an aberrant polypeptide.

In another aspect, the invention contemplates the use of an antigen-presenting cell for modulating the level or functional activity of an aberrant polypeptide product of a gene selected from PSA or KLK2, or a variant or derivative of the polypeptide product, wherein the antigen-presenting cell expresses a processed form of the aberrant polypeptide product for presentation to, and modulation of, T cells.

According to yet another aspect, the invention provides a method for the treatment and/or prophylaxis of a prostate cancer, comprising administering to a patient in need of such treatment an effective amount of an agent as broadly described above or of an antigen-presenting cell as broadly described above and optionally a pharmaceutically acceptable carrier.

### BRIEF DESCRIPTION OF THE DRAWINGS

FIG. 1 is a schematic representation of the structure of the wild-type PSA mRNA and variants, PSA RP2 transcript 1, PSA RP2 transcript 2, PSA 525 and PSA (Schulz). Exons (1-5) of the PSA gene from which the mRNAs are derived, are denoted as coloured boxes, intronic insertions are represented as solid lines.

FIG. 2 is a schematic representation of the structure of the wild type KLK2 mRNA and variant, 10A. Exons (1-5) of the KLK2 gene from which the mRNAs are derived, are denoted as coloured boxes, intronic insertions are represented as solid lines.

FIG. 3 is a photographic representation of ethidium bromide stained TAE-agarose gel of RT-PCRs for the mRNA transcripts, PSA and KLK2 wild type, PSA RP2 transcripts 1&2 (T1 and T2, respectively), PSA 525, PSA (Schulz) and KLK2 10A amplified in 9 prostate cancer and 4 BPH specimens. The sizes of PCR products, in base pairs (bp), are indicated on the right. (Key: M=molecular weight marker IX (Roche), N=no cDNA control).

FIG. 4 is a graphical representation of real-time PCR analysis of cancer samples (PCa) and BPH samples for the PSA variant mRNA transcripts: PSA wild type (24 PCa, 27 BPH samples), PSA-RP2 (19 PCa, 14 BPH samples), PSA-RP1 (24 PCa, 28 BPH samples) and the PSA (Schulz) transcript (24 PCa, 28 BPH samples). Each sample was assayed in duplicate, two times. Data are presented as the ratio of the averaged transcript copy number divided by the averaged β₂-microglobulin transcript number from each individual sample, with standard error of the mean (bars). Student's t-test analysis yielded only PSA-RP2 as significantly different between the two groups (*P<0.05).

FIG. 5 is a graphical representation of real-time PCR analysis of 21 cancer samples and 20 BPH samples for the KLK2 wild type transcript, and 20 cancer samples and 21 BPH samples for the KLK2 variant mRNA transcript, KLK2-10A. Each sample was assayed in duplicate, two times. Data are presented as the ratio of the averaged transcript copy number divided by the averaged 12-microglobulin transcript number from each individual sample, with standard error of the mean (bars). Student's t-test analysis yielded KLK2-10A as highly significantly different between the two groups (*P<0.001).

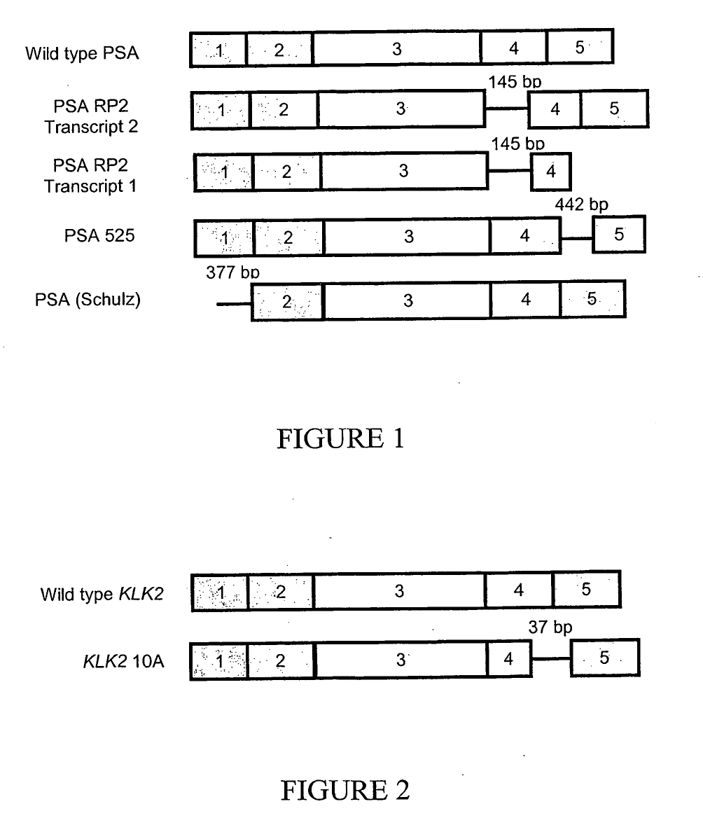

### BRIEF DESCRIPTION OF THE SEQUENCES: SUMMARY TABLE

TABLE A

| SEQUENCE ID | | |
|---|---|---|
| NUMBER | SEQUENCE | LENGTH |
| SEQ ID NO: 1 | Nucleotide sequence corresponding to human PSA RP2 | 1603 nts |
| | transcript 2 mRNA as set forth in GenBank Accession No. | |
| | AJ310938 | |
| SEQ ID NO: 2 | Prepro-PSA RP2 amino acid sequence encoded by SEQ ID | 180 aa |
| | NO: 1 | |
| SEQ ID NO: 3 | Nucleotide sequence corresponding to human PSA RP2 | 492 nts |
| | transcript ½ mRNA encoding pro-PSA RP2 | |
| SEQ ID NO: 4 | Pro-PSA RP2 amino acid sequence encoded by SEQ ID NO: 3 | 163 aa |
| SEQ ID NO: 5 | Nucleotide sequence corresponding to human PSA RP2 | 471 nts |
| | transcript ½ mRNA encoding mature PSA RP2 | |
| SEQ ID NO: 6 | Mature PSA RP2 amino acid sequence encoded by SEQ ID | 156 aa |
| | NO: 5 | |
| SEQ ID NO: 7 | Nucleotide sequence corresponding to human PSA RP2 | 708 bp |
| | transcript 1 mRNA as set forth in GenBank Accession No. | |
| | AJ310937 | |
| SEQ ID NO: 8 | PSA RP2 amino acid sequence which is different to wild- | 16 aa |
| | type PSA | |
| SEQ ID NO: 9 | Nucleotide sequence corresponding to human KLK2 10A | 1541 nts |
| | mRNA as set forth in GenBank Accession No. S39329 | |
| SEQ ID NO: 10 | K2 10A polypeptide encoded by SEQ ID NO: 9 | 223 aa |
| SEQ ID NO: 11 | Nucleotide sequence corresponding to human KLK 10A | 621 |
| | mRNA encoding pro-K2 10A | |
| SEQ ID NO: 12 | Pro-K2 10A amino acid sequence encoded by SEQ ID NO: | 206 aa |
| | 11 | |
| SEQ ID NO: 13 | Nucleotide sequence corresponding to human KLK 10A | 600 nts |
| | mRNA encoding mature K2 10A | |
| SEQ ID NO: 14 | Mature K2 10A amino acid sequence encoded by SEQ ID | 199 aa |
| | NO: 13 | |
| SEQ ID NO: 15 | K2 10A amino acid sequence which is different to wild-type | 13 aa |
| | K2 | |
| SEQ ID NO: 16 | PSA wild-type forward primer of Table 1 | 27 nts |
| SEQ ID NO: 17 | PSA wild-type reverse primer of Table 1 | 22 nts |
| SEQ ID NO: 18 | PSA RP2 transcripts 1 and 2 forward primer of Table 1 | 27 nts |
| SEQ ID NO: 19 | PSA RP2 transcripts 1 and 2 reverse primer of Table 1 | 21 nts |
| SEQ ID NO: 20 | PSA 525 forward primer of Table 1 | 21 nts |
| SEQ ID NO: 21 | PSA 525 reverse primer of Table 1 | 21 nts |
| SEQ ID NO: 22 | PSA (Schulz) forward primer of Table 1 | 21 nts |
| SEQ ID NO: 23 | PSA (Schulz) reverse primer of Table 1 | 27 nts |
| SEQ ID NO: 24 | KLK2 wild type forward primer of Table 1 | 22 nts |
| SEQ ID NO: 25 | KLK2 wild type reverse primer of Table 1 | 21 nts |
| SEQ ID NO: 26 | KLK2 10A forward primer of Table 1 | 21 nts |
| SEQ ID NO: 27 | KLK2 10A reverse primer of Table 1 | 27 nts |
| SEQ ID NO: 28 | β2-microgloblin forward primer of Table 1 | 21 nts |
| SEQ ID NO: 29 | β2-microgloblin reverse primer of Table 1 | 21 nts |
| SEQ ID NO: 30 | PSA wild-type forward primer of Table 2 | 21 nts |
| SEQ ID NO: 31 | PSA wild-type reverse primer of Table 2 | 21 nts |
| SEQ ID NO: 32 | PSA RP2 transcripts 1 and 2 forward primer of Table 2 | 21 nts |
| SEQ ID NO: 33 | PSA RP2 transcripts 1 and 2 reverse primer of Table 2 | 22 nts |
| SEQ ID NO: 34 | PSA 525 forward primer of Table 2 | 21 nts |
| SEQ ID NO: 35 | PSA 525 reverse primer of Table 2 | 27 nts |
| SEQ ID NO: 36 | PSA (Schulz) forward primer of Table 2 | 21 nts |
| SEQ ID NO: 37 | PSA (Schulz) reverse primer of Table 2 | 21 nts |
| SEQ ID NO: 38 | KLK2 wild type forward primer of Table 2 | 22 nts |
| SEQ ID NO: 39 | KLK2 wild type reverse primer of Table 2 | 21 nts |
| SEQ ID NO: 40 | KLK2 10A forward primer of Table 2 | 21 nts |
| SEQ ID NO: 41 | KLK2 10A reverse primer of Table 2 | 27 nts |
| SEQ ID NO: 42 | β2-microgloblin forward primer of Table 2 | 21 nts |
| SEQ ID NO: 43 | β2-microgloblin reverse primer of Table 2 | 21 nts |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “aberrant polynucleotide” refers to a polynucleotide which differs from a “normal” reference polynucleotide by the substitution, deletion and/or addition of at least one nucleotide and which correlates with the presence or risk of a condition such as cancer.

The term “aberrant expression product” refers to expressed polynucleotides or polypeptide, which differ from a “normal” reference polynucleotide or polypeptide by the substitution, deletion and/or addition of one or more nucleotides or amino acid residues, and which correlate with the presence or risk of a condition such as cancer.

The term “aberrant polypeptide” refers to a polypeptide which differs from a “normal” reference polypeptide by the substitution, deletion and/or addition of at least one amino acid residue and which correlates with the presence or risk of a condition such as cancer.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in a mammal to which it is administered, wherein the response includes the production of elements which specifically bind the polypeptide or fragment thereof.

The term “biological sample” as used herein refers to a sample that may be extracted, untreated, treated, diluted or concentrated from a patient. The biological sample is preferably selected from cell and tissue samples including cells and tissue from the prostate, lymph and bone. The biological sample may also be a fluid selected from the group consisting of whole blood, serum, plasma, saliva, urine, sweat, ascitic fluid, peritoneal fluid, synovial fluid, cerebrospinal fluid, skin biopsy, and the like. Preferably, the biological sample includes serum, whole blood, plasma and lymph or fractionated portion thereof as well as other circulatory fluid and saliva, mucus secretion and respiratory fluid.

By “biologically active fragment” is meant a fragment of a full-length parent polypeptide which fragment retains the activity of the parent polypeptide. For example, a biologically active fragment of a K2 polypeptide will have the ability to elicit the production of elements that specifically bind to that polypeptide. As used herein, the term “biologically active fragment” includes deletion variants and small peptides, for example of at least 10, preferably at least 20 and more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthesis Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount”, in the context of treating or preventing a condition is meant the administration of that amount of active substance to an individual in need of such treatment or prophylaxis, either in a single dose or as part of a series, that is effective for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linking” a promoter to a polynucleotide is meant placing the polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the polynucleotide, which is approximately the same as the distance between that promoter and the gene it controls in its natural setting; i.e.: the gene from which the promoter is derived. As is known in the art, some variation in this distance can be accommodated without loss of function.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, avians, reptiles).

By “pharmaceutically acceptable carrier” is meant a solid or liquid filler, diluent or encapsulating substance that can be safely used in topical or systemic administration to a animal, preferably a mammal including humans.

The term “polynucleotide” or “nucleic acid” as used herein designates mRNA, RNA, cRNA, cDNA or DNA. The term typically refers to oligonucleotides greater than 30 nucleotide residues in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but can alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridise and serve as a site for the initiation of synthesis. By “substantially complementary”, it is meant that the primer is sufficiently complementary to hybridise with a target polynucleotide. Preferably, the primer contains no mismatches with the template to which it is designed to hybridise but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, ie. through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (ie. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (ie. resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (eg. A, T, C, G, I) or the identical amino acid residue (eg. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (ie. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Similarity” refers to the percentage number of amino acids that are identical or constitute conservative substitutions. For example, a conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mis-matching between hybridised sequences. Those skilled in the art will recognise that other physical and chemical parameters in the hybridisation and wash stages can also be altered to affect the outcome of a detectable hybridisation signal from a specific level of homology between target and probe sequences.

The term “variant” is used herein to refer to polynucleotides and polypeptides that differ from a reference polynucleotide or polypeptide by the addition, deletion or substitution of at least one subunit. Variant polynucleotides include polynucleotides displaying substantial sequence identity with a reference polynucleotide sequence or polynucleotides that hybridise with a reference sequence under stringent conditions that are defined hereinafter. Variant polynucleotides also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. Variant polynucleotides also include naturally occurring allelic variants. Variant polypeptides encompass polypeptides in which one or more amino acids have been added or deleted, or replaced with different amino acids. It is well understood in the art that some amino acids may be changed to others with broadly similar properties without changing the nature of the activity of the polypeptide (conservative substitutions).

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, ie. a vector that exists as an extrachromosomal entity, the replication of which is independent of chromosomal replication, eg. a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®)) and the hph gene which confers resistance to the antibiotic hygromycin B.

As used herein, underscoring or italicising the name of a gene shall indicate the gene, in contrast to its protein product, which is indicated in the absence of any underscoring or italicising. By way of example: (1) “KLK2” shall mean the KLK2 gene, whereas “K2” shall indicate the protein product of the “KLK2” gene; and (2) “PSA” shall mean the PSA gene, whereas “PSA” shall indicate the protein product of the “PSA” gene.

2. Methods of Detecting Abberant PSA or KLK2 Expression

The present invention is predicated in part on the discovery that patients with prostate cancer produce different levels of aberrant transcripts, and presumably of aberrant polypeptides, corresponding to the PSA and KLK2 genes, compared to patients with benign prostate hyperplasia (BPH). Thus, the invention features a method for detecting the presence or diagnosing the risk of prostate cancer in a patient, comprising detecting in a biological sample obtained from the patient a level or functional activity of an aberrant expression product of a gene selected from PSA or KLK2, which correlates with the presence or risk of prostate cancer.

In a preferred embodiment, the aberrant PSA expression product is selected from an aberrant PSA transcript or polypeptide product thereof, wherein the transcript comprises an intronic insertion and preferably a 145 nt insertion from intron 3 of the wild-type PSA gene, which insertion is characteristic of PSA RP2 transcripts 1 or 2. In an especially preferred embodiment, the aberrant PSA expression product is selected from PSA RP2 transcript 1, PSA RP2 transcript 2, or a polypeptide product of these. Both these PSA RP2 transcripts have a 145 nt insertion from intron 3 of the PSA gene. The PSA RP2 transcript 1, which was first characterised by Riegman et al. (1988, Biochem. Biophys. Res. Comm. 155: 181-188), is smaller than the wild-type PSA transcript (0.7 kb as compared to 1.6 kb for wild-type PSA) and has an early polyadenylation signal sequence in exon 4. The nucleotide sequence corresponding to the PSA RP2 transcript 1 is presented in SEQ ID NO: 7. The PSA RP2 transcript 2, which was later identified by Heuźe-Vourc'h et al., (2001, Eur. J. Biochem. 268(1): 4408-4413), is 1.6 kb long and comprises 895 nts of additional 3′ UTR. The nucleotide sequence corresponding to the PSA RP2 transcript 2 is set forth in SEQ ID NO: 1. Both transcripts 1 and 2 produce a 180 aa protein, due to an in-frame stop codon that results in the truncated prepro-PSA variant. The amino acid sequence of this protein variant is set forth in SEQ ID) NO: 2. Each of PSA RP2 transcripts 1 and 2 comprise the nucleotide sequences set forth in SEQ ID NO: 3 and 5, which code for the pro- and mature forms of PSA RP2, respectively. The catalytic triad is not conserved, thus making the prepro-, pro- and mature protein products of PSA RP2, as set forth in SEQ ID NO: 2, 4 or 6, respectively, devoid of serine protease activity or at least having reduced serine protease activity relative to wild-type PSA.

In another preferred embodiment, the aberrant KLK2 expression product is selected from an aberrant KLK2 transcript or polypeptide product thereof, wherein the transcript comprises an intronic insertion and preferably a 37 nt insertion from intron 4 of the wild-type KLK2 gene, which insertion is characteristic of the KLK2 10A transcript. In an especially preferred embodiment, the aberrant KLK2 expression product is selected from the KLK2 10A variant transcript or a polypeptide product thereof. The KLK2 10A transcript, which was first identified by Riegman et al. (1991, Molecular and Cellular Endocrinology 76: 181-190), is differentially spliced at intron 4 of the KLK2 gene, resulting in a 37 bp intronic insertion. The nucleotide sequence corresponding to the human KLK2 10A mRNA is presented in SEQ ID NO: 9 and its 233 aa protein product (prepro-K2 10A) is presented in SEQ ID NO: 10. At amino acid 211, the K2 10A product deviates from the wild-type K2 sequence because the intronic insertion results in a shift in the reading frame. The KLK2 10A transcript comprises nucleotide sequences set forth in SEQ ID NO: 11 and 13, which encode respectively the pro- and mature forms of K2 10A, are set forth in SEQ ID NO: 12 and 14. The catalytic triad is not conserved, thus making this aberrant protein product devoid of serine protease activity or at least having reduced serine protease activity relative to wild-type K2.

The correlation with the presence or risk of prostate cancer is made for example when the level or functional activity of the PSA or KLK2 aberrant expression product in the biological sample differs by at least 10%, more preferably by at least 50%, even more preferably by at least 100%, even more preferably by at least 200%, even more preferably by at least 400%, even more preferably by at least 600% and still even more preferably by at least 1000% compared to the level or functional activity of the expression product that correlates with the presence or risk of BPH. In a preferred embodiment, the level or functional activity of the aberrant expression product in the biological sample is at least 10%, more preferably at least 50%, even more preferably at least 100%, even more preferably at least 200%, even more preferably at least 400%, even more preferably at least 600% and still even more preferably at least 1000% higher than the level or functional activity of the expression product in a biological sample obtained from patients with BPH. Thus, the presence or risk of prostate cancer is diagnosed when an aberrant PSA or KLK2 expression product is expressed at a detectably higher level compared to the level at which it is expressed in patients with BPH. In a preferred embodiment of this type, the method comprises detecting a level or functional activity of an aberrant expression product of an aberrant PSA or KLK2 expression product, which is elevated relative to a reference level or functional activity of the aberrant expression product, which correlates with BPH.

Thus, it will be desirable to qualitatively or quantitatively determine the levels of an aberrant polypeptide or aberrant transcript as defined herein in a biological sample, which include but is not limited to, a cell or tissue sample or a biological fluid. In one embodiment, the biological sample is a cell, which is preferably of prostatic origin. The cell may be isolated from a tissue source including, but not limited to prostate tissue, lymph tissue, and bone tissue, or from a biological fluid which is preferably selected from seminal fluid, whole blood, serum or lymphatic fluid. In another embodiment, the expression product (e.g., an aberrant polypeptide) is present in the extracellular compartment and is therefore present as a soluble entity in a biological fluid. In particular embodiments of the present invention, the presence in fluid or tissue of non-prostatic origin of a prostate cancer-correlating level or functional activity of the aberrant expression product indicates the presence or risk of metastatic prostate cancer in the patient. By contrast, the presence or risk of organ-confined prostate cancer is suggested if that level or functional activity is absent in fluid or tissue of non-prostatic origin.

2.1 Genetic Diagnosis

One embodiment of the instant invention comprises a method for detecting an increase in the expression of an aberrant PSA polynucleotide or of an aberrant KLK2 polynucleotide by qualitatively or quantitatively determining aberrant PSA or KLK2 transcripts in a cell (e.g., a cell of prostatic origin). Exemplary nucleic acid sequences for such transcripts are set forth in the enclosed Sequence Listing and are summarised in TABLE A supra. Nucleic acid used in polynucleotide-based assays can be isolated from cells contained in the biological sample, according to standard methodologies (Sambrook, et al., “Molecular Cloning. A Laboratory Manual”, Cold Spring Harbor Press, 1989; Ausubel et al., “Current Protocols in Molecular Biology”, John Wiley & Sons Inc, 1994-1998). The nucleic acid is preferably whole cell RNA or fractionated portion thereof, which if desired, may be to converted to a complementary DNA (cDNA). In one embodiment, the RNA is whole cell RNA; in another, it is poly-A RNA. In a preferred embodiment, the nucleic acid is amplified by a nucleic acid amplification technique. Suitable nucleic acid amplification techniques are well known to the skilled addressee, and include the polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl. Chem., A31(1): 1355-1376).

Following detection, one may compare the results seen in a given patient with a control reaction or a statistically significant reference group of patients with BPH or prostate cancer. In this way, it is possible to correlate the amount of a aberrant transcript detected with the progression or severity of the disease.

1.1.1 Primers and Probes

Primers may be provided in double-stranded or single-stranded form, although the single-stranded form is preferred. Probes, while perhaps capable of priming, are designed to bind to a target DNA or RNA and need not be used in an amplification process. In preferred embodiments, the probes or primers are labelled with radioactive species ³²P, ¹⁴C, ³⁵S, ³H, or other label), with a fluorophore (rhodamine, fluorescein) or a chemillumiscent label (luciferase).

1.1.2 Template Dependent Amplification Methods

A number of template dependent processes are available to amplify the aberrant transcripts that may be present in a given sample. These process will generally comprise reverse transcribing RNA into cDNA. Methods of reverse transcribing RNA into cDNA are well known and described in Sambrook et al., 1989. Alternative methods for reverse transcription utilise thermostable, RNA-dependent DNA polymerases. These methods are described in WO 90/07641. The cDNA is then amplified using a suitable nucleic acid amplification technique.

An exemplary nucleic acid amplification technique is the polymerase chain reaction (referred to as PCR) which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Polymerase chain reaction methodologies are well known in the art. Briefly, in PCR, two primer sequences are prepared that are complementary to regions on opposite complementary strands of a target sequence. An excess of deoxynucleoside triphosphates are added to a reaction mixture along with a DNA polymerase, e.g., Taq polymerase. If the target sequence is present in a sample, the primers will bind to the target and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention, Walker et al., (1992, Proc. Nail. Acad. Sci. U.S.A 89: 392-396).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridised to DNA that is present in a sample. Upon hybridisation, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 disclose a nucleic acid amplification process involving cyclically synthesising single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase D, resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridisation of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl. Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989, Genomics 4: 560).

In an especially preferred embodiment, the level of an aberrant transcript is quantified using a nucleic acid amplification technique that quantifies an aberrant transcript in real-time. For example, RNA or DNA may be quantitated using the Real-Time PCR technique (Higuchi, 1992, et al., Biotechnology, 10: 413417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesised from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA.

Therefore, the first condition that must be met before the relative abundance of a RNA or DNA species can be determined by Real-Time PCR for a collection of RNA or DNA populations is that the concentrations of the amplified PCR products must be sampled when the PCR reactions are in the linear portion of their curves.

The second condition that must be met for an real-time-PCR experiment to successfully determine the relative abundance of a particular mRNA species is that relative concentrations of the amplifiable cDNAs must be normalised to some independent standard. The goal of a Real-Time PCR experiment is to determine the abundance of a particular RNA or DNA species relative to the average abundance of all RNA or DNA species in the sample.

1.1.3 Northern Blotting

Northern blotting techniques are well known to those of skill in the art and involve the use of RNA or cDNA as a target. Briefly, a probe is used to target a cDNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labelled) under conditions that promote denaturation and rehybridization. Because the probe is designed to base pair with the target, the probe will binding a portion of the target sequence under renaturing conditions. Unbound probe is then removed, and detection is accomplished as described above.

1.1.4 Identification Methods

The aberrant polynucleotides of the invention or amplification products relating thereto are typically analysed visually or by other means in order to confirm amplification of the target-gene(s) sequences. One typical visualisation method involves staining of a gel with for example, a fluorescent dye, such as ethidium bromide or Vistra Green and visualisation under UV light. Alternatively, if the amplification products are integrally labelled with radio- or fluorometrically-labelled nucleotides, the amplification products can then be exposed to x-ray film or visualised under the appropriate stimulating spectra, following separation.

In one embodiment, visualisation is achieved indirectly, using a nucleic acid probe. Following separation of amplification products, a labelled, nucleic acid probe is brought into contact with the amplified target sequence. The probe preferably is conjugated to a chromophore but may be radiolabelled. In another embodiment, the probe is conjugated to a binding partner, such as an antibody or biotin, where the other member of the binding pair carries a detectable moiety. In other embodiments, the probe incorporates a fluorescent dye or label. In yet other embodiments, the probe has a mass label that can be used to detect the molecule amplified. Other embodiments also contemplate the use of Taqman™ and Molecular Beacon™. probes. In still other embodiments, solid-phase capture methods combined with a standard probe may be used as well.

The type of label incorporated in PCR products is dictated by the method used for analysis. When using capillary electrophoresis, microfluidic electrophoresis, TPLC, or LC separations, either incorporated or intercalated fluorescent dyes are used to label and detect the nucleic acid amplification products. Samples are detected dynamically, in that fluorescence is quantitated as a labelled species moves past the detector. If any electrophoretic method, BPLC, or LC is used for separation, products can be detected by absorption of UV light, a property inherent to DNA and therefore not requiring addition of a label. If polyacrylamide gel or slab gel electrophoresis is used, primers for the nucleic acid amplification can be labelled with a fluorophore, a chromophore or a radioisotope, or by associated enzymatic reaction. Enzymatic detection involves binding an enzyme to primer, e.g., via a biotin:avidin interaction, following separation of nucleic acid amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analysed more than once by probing, stripping the blot, and then re-probing. If nucleic acid amplification products are separated using a mass spectrometer no label is required because nucleic acids are detected directly.

A number of the above separation platforms can be coupled to achieve separations based on two different properties. For example, some of the nucleic acid amplification primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

1.1.5 Kit Components

All the essential materials and reagents required for qualitatively or quantitatively determining the level of an aberrant expression product as herein defined may be assembled together in a kit. The kits may optionally include appropriate reagents for detection of labels, positive and negative controls, washing solutions, dilution buffers and the like. For example, a nucleic acid-based detection kit may include (i) an aberrant polynucleotide according to the invention (which may be used as a positive control), and/or (ii) an oligonucleotide primer which corresponds or is complementary to the aberrant polynucleotide. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits will also generally comprise, in suitable means, distinct containers for each individual reagent and enzyme as well as for each primer or probe.

1.1.6 Chip Technologies

Also contemplated by the present invention are chip-based DNA technologies such as those described by Hacia et al. (1996, Nature Genetics 14: 441-447) and Shoemaker et al. (1996, Nature Genetics 14: 450-456). Briefly, these techniques involve quantitative methods for analysing large numbers of target sequences rapidly and accurately. By tagging target sequences with oligonucleotides or using fixed probe arrays, one can employ chip technology to segregate target molecules as high density arrays and screen these molecules on the basis of hybridisation. See also Pease et al. (1994, Proc. Natl. Acad. Sci. U.S.A. 91: 5022-5026); Fodor et al. (1991, Science 251: 767-773). The present invention contemplates the preparation of a high-density array of primers on a chip (or on any other solid surface) and conduct the nucleic acid amplifications on this solid-phase.

1.2 Protein-Based Diagnostics

1.2.1 Antigen-Binding Molecules

Antigen-binding molecules that are immuno-interactive with an aberrant polypeptide product of a gene selected from PSA or KLK2 can be used in measuring an increase in the level of these polypeptide products. Thus, the present invention also contemplates antigen-binding molecules that bind specifically to such polypeptides and especially to PSA RP2 or K2 10A, which preferably comprise the sequences set forth in SEQ ID NO: 2, 4, 6, 10, 12 or 14. In especially preferred embodiments, the antigen-binding molecules are immuno-interactive specifically with SEQ ID NO: 8 or 15.

1.2.2 Immunodiagnostic Assays

The above antigen-binding molecules have utility in measuring directly or indirectly modulation of expression in healthy and diseased states, through techniques such as ELISAs and Western blotting. Illustrative assay strategies which can be used to detect a target polypeptide of the invention include, but are not limited to, immunoassays involving the contacting of an antigen-binding molecule to the target polypeptide (e.g., a PSA RP2 or K2 10A polypeptide) in the sample, and the detection of a complex comprising the antigen-binding molecule and the target polypeptide. Preferred immunoassays are those that can measure the level or functional activity of a target molecule of the invention. Typically, an antigen-binding molecule that is immuno-interactive with a target polypeptide of the invention is contacted with a biological sample suspected of containing the target polypeptide. The concentration of a complex comprising the antigen-binding molecule and the target polypeptide is measure in and the measured complex concentration is then related to the concentration of target polypeptide in the sample. Consistent with the present invention, the presence of a concentration of the target polypeptide which correlates with the presence or risk of prostate cancer is indicative of that presence or risk.

Any suitable technique for determining formation of an antigen-binding molecule-target antigen complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to Coligan et al. (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue or fluid as described above.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:
(a) direct attachment of the reporter molecule to the antigen-binding molecule;
(b) indirect attachment of the reporter molecule to the antigen-binding molecule; i.e., attachment of the reporter molecule to another assay reagent which subsequently binds to the antigen-binding molecule; and
(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

It will be well understood that other means of testing target polypeptide (e.g., K2 10A or PSA RP2) levels are available, including, for instance, those involving testing for an altered level of aberrant polypeptide binding activity to an integrin, or Western blot analysis of aberrant polypeptide levels in tissues, cells or fluids using anti-aberrant polypeptide antigen-binding molecule, or assaying the amount of antigen-binding molecule or other aberrant polypeptide binding partner which is not bound to a sample, and subtracting from the total amount of antigen-binding molecule or binding partner added.

3. Identification of Target Molecule Modulators

The invention also features a method of screening for an agent that modulates the level or functional activity of a target molecule comprising an aberrant expression product of a gene selected from PSA and KLK2. The screening method comprises contacting a preparation comprising at least a portion of an aberrant polypeptide encoded by the gene, or a variant or derivative thereof, or a genetic sequence that modulates the expression of the gene, with the agent and detecting a change in the level or functional activity of the polypeptide or variant or derivative, or of a product expressed from the genetic sequence.

In accordance with the present invention, agents that modulate these target molecules are useful for treating or modulating tumorigenesis and especially for preventing prostate cancer. Any suitable assay for detecting, measuring or otherwise determining modulation of tumorigenesis is contemplated by the present invention. Assays of a suitable nature are known to persons of skill in the art. It will be understood, in this regard, that the present invention is not limited to the use or practice of any one particular assay for determining a the activity.

Tumorigenesis is typically associated with promotion of cell proliferation, cell migration, invasion and/or motility. Generally, for cell proliferation, cell number is determined, directly, by microscopic or electronic enumeration, or indirectly, by the use of chromogenic dyes, incorporation of radioactive precursors or measurement of metabolic activity of cellular enzymes. An exemplary cell proliferation assay comprises culturing cells in the presence or absence of a test compound, and detecting cell proliferation by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65: 55-63).

Cancer or tumour markers are known for a variety of cell or tissue types. Cells or tissues expressing cancer or tumour markers may be detected using monoclonal antibodies, polyclonal antisera or other antigen-binding molecules that are immuno-interactive with these markers or by using nucleic acid analysis techniques, including, for example, detecting the level or presence of marker-encoding polynucleotides.

Prostate cancer death results primarily from invasion into the organ surrounds and metastasis. The first step to invasion is the degradation of the extracellular matrix (ECM) and subsequent migration from the primary tumour to distant sites. As well as its fibronectin degrading-role in the semen (Lilja, 1985, Journal of Clinical Investigation 76: 1899-1903), PSA has been reported to degrade fibronectin and laminin, two major proteins incorporated into the ECM (Webber et al., 1995, Clinical Cancer Research 1: 1089-1094). Thus, PSA may be directly and/or indirectly involved in localised proteolysis of ECM proteins in tumour cell invasion in vivo. Accordingly, the present invention contemplates the use of any suitable method for assaying tumour cell invasion. For example, the chemo-invasion assay (Albini et al., 1987, Cancer Research 47: 3239-3245) may be used, which involves coating the membranous portion of a tissue culture insert with Matrigel, a synthetic form of extracellular matrix (ECM). Supernantants of NIH 3T3 murine fibroblasts are often used as chemo-attractants, alternatively, 20% foetal calf serum (FCS) can be used, and are placed in the lower chamber. Cells are plated and allowed to grow for 6-72 hours at 37° C., depending on invasive potential. Cells on the upper side of the membrane are removed, and those that migrate to the under-surface are fixed, stained with crystal violet, then solubilised using acetic acid. The extracted dye is then read at 600 nm.

Another useful assay for analysing the tumorigenesis of the aberrant expression products of the present invention is the migration/wound assay as for example disclosed by Sato and Rifkin. (1988, J. Cell Biol. 107: 1589-1598), Verrall et al. (1999, Cancer Letters 145: 79-83) and Fortier et al. (1999, JNCI 91: 1635-40). In one embodiment of this assay, cells are grown in gelatin-coated tissue culture dishes or other appropriate tissue culture containers and incubated at 37° C. for 24-72 hours. After incubation, cells are wounded by a sterile single-edged razor blade or pipette, which is drawn across the plate surface thus creating a cell-free area, the ‘wound’. Remaining cells are subsequently washed in phosphate-buffered saline (PBS) and further incubated in growth media. Wounded cells are then exposed to growth factors, etc, for a number of hours. Cells are subsequently fixed and stained. Migration is quantified by counting the number of cells that migrate from the wound edge into the denuded area.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of the aberrant expression products of the present invention are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues. Screening may also be directed to known pharmacologically active compounds and chemical analogues thereof.

Candidate modulators of the present invention include agonists and especially antagonists of the aberrant expression products defined herein. Antagonists of the aberrant transcripts of the invention include antisense molecules, ribozymes and co-suppression molecules. Agonists include molecules which increase promoter activity or interfere with negative mechanisms. Agonists of the aberrant expression products include molecules which overcome any negative regulatory mechanism. Antagonists of the aberrant polypeptides of the invention include antibodies and inhibitor peptide fragments.

In a series of preferred embodiments, the present invention provides assays for identifying small molecules or other compounds (i.e., modulatory agents) which are capable of inducing or inhibiting the level or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level or functional activity of the target molecule, any change in the level from an established baseline may be detected using any of the techniques described above and well known in the art. In particularly preferred embodiments, the cells are epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that inhibit the activity of an aberrant polypeptide is provided in which a purified preparation of that polypeptide is contacted with a candidate agent, and the level of aberrant polypeptide's activity is measured by a suitable assay. For example, an aberrant polypeptide inhibitor can be identified by measuring the ability of a candidate agent to decrease aberrant polypeptide activity in a cell (e.g., an ovarian cell). In this method, a cell that is capable of expressing an aberrant transcript is exposed to, or cultured in the presence and absence of, the candidate agent, and an activity associated with tumorigenesis an is detected. An agent tests positive if it inhibits this activity.

In a preferred embodiment, the agent, which is identifiable for example by the above methods, inhibits, abrogates or otherwise reduces the expression of an aberrant expression product as herein defined for the treatment and/or prophylaxis of prostate cancer. For example, agents that may be used to reduce or abrogate gene expression include, but are not restricted to, oligoribonucleotide sequences, including anti-sense RNA and DNA molecules and ribozymes, that function to inhibit the translation, for example, of KLK2 10A encoding mRNA. Anti-sense RNA and DNA molecules act to directly block the translation of mRNA by binding to targeted mRNA and preventing protein translation. In regard to antisense DNA, oligodeoxyribonucleotides derived from the intronic insertion region of an aberrant transcript of the invention, are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of KLK2 10A or PSA RP2 transcript 1 or 2 RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of nucleic acid molecules. These include techniques for chemically synthesising oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, anfisense cDNA constructs that synthesise antisense RNA constitutively or inducibly, depending on the promoter used, can be introduced stably into cell lines.

Various modifications to nucleic acid molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Other agents that may be used to decrease the level or functional activity of an aberrant expression product of the invention include RNA molecules that mediate RNA interference (RNAi) of a target gene or gene transcript. RNAi refers to interference with or destruction of the product of a target gene by introducing a single stranded, and typically a double stranded RNA (dsRNA), that is homologous to an aberrant PSA or KLK2 transcript of the invention. Thus, in one embodiment, dsRNA per se and especially dsRNA-producing constructs corresponding to at least a portion of a an aberrant PSA or KLK2 transcript may be used to decrease its level aor functional activity. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are preferably at least about 100 nucleotides in length. RNA-encoding constructs that express a single RNA designed to form a dsRNA via internal folding are preferably at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors.

In another embodiment, RNA molecules of about 21 to about 23 nucleotides, which direct cleavage of specific mRNA to which they correspond, as for example described by Tuschl et al. in U.S. patent application No. 20020086356, can be utilised for mediating RNAi. Such 21-23 nt RNA molecules can comprise a 3′ hydroxyl group, can be single-stranded or double stranded (as two 21-23 nt RNAs) wherein the dsRNA molecules can be blunt ended or comprise overhanging ends (e.g., 5′, 3′).

4. Antigen-Presenting Cell Embodiments

The invention further provides a composition of matter for eliciting a humoral and a cellular immune response against an aberrant polypeptide product of a gene selected from PSA or KLK2, or a variant or derivative of the polypeptide product. The composition generally comprises antigen-presenting cells which express a processed form of an antigen comprising the aberrant polypeptide product, for presentation to, and modulation of, T cells. Antigen-primed antigen-presenting cells may be prepared by a method including contacting antigen-presenting cells with an antigen selected from the aberrant polypeptide product or a polynucleotide from which the aberrant polypeptide product is expressible, for a time and under conditions sufficient to permit the antigen to be internalised by the antigen-presenting cells, and culturing the antigen-containing antigen-presenting cells for a time and under conditions sufficient for the antigen to be processed for presentation by the antigen-presenting cells. Alternatively, antigen-primed antigen-presenting cells may be prepared by transfecting antigen-presenting cells with a polynucleotide from which the aberrant polypeptide product is expressible, and culturing the polynucleotide-containing antigen-presenting cells for a time and under conditions sufficient for expression of the polynucleotide to produce the aberrant polypeptide product and for processing of the aberrant polypeptide product for presentation to, and modulation of, T cells. The antigen-presenting cells may be selected from dendritic cells, macrophages and B cells. In preferred embodiments of the invention, the antigen-presenting cells are dendritic cells.

4.1 Sources of Dendritic Cells

Dendritic cells can be isolated by methods known to those of skill in the art. Suitably, mammalian and preferably human dendritic cells are used from an appropriate tissue source, which is suitably blood or bone marrow. Dendritic cell precursors, from which the immature dendritic cells for use in antigen internalisation according to the invention, are present in blood as peripheral blood mononuclear cells (PBMCs). Although most easily obtainable from blood, the precursor cells may also be obtained from any tissue in which they reside, including bone marrow and spleen tissue. Peripheral blood precursors may be purified using monoclonal antibodies, density gradients or centrifugation or any combination of these. Circulating frequency may be increased in vivo using flt-3 ligand. When cultured in the presence of cytokines such as a combination of GM-CSF and IL-4 or IL-13 as described below, the non-proliferating precursor cells give rise to immature dendritic cells for use in this invention.

An exemplary method for culturing pluripotential PBMCs to produce immature dendritic cells is described by Albert et al. (International Publication WO 99/42564). In this respect, cultures of immature dendritic cells, ie. antigen-capturing phagocytic dendritic cells, may be obtained by culturing non-proliferating precursor cells (PBMCs) in the presence of cytokines which promote their differentiation. A combination of GM-CSF and IL4 produces significant quantities of the immature dendritic cells, ie. antigen-capturing phagocytic or internalisation-competent dendritic cells. Other cytokines that promote differentiation of precursor cells into immature dendritic cells include, but are not limited to, IL-13.

Maturation of dendritic cells requires the addition to the cell environment, preferably the culture medium, of a dendritic cell one or more maturation factors which may be selected from monocyte conditioned medium and/or factors including TNF-α; IL-6, IFN-α and IL-1. Alternatively, a mixture of necrotic cells or necrotic cell lysate may be added to induce maturation. Maturation can be induced in vitro using plastic adherence, cytokines, LPS, bacteria, DNA containing CpG repeats, RNA or polyIC, CD40-ligation, necrotic cells. In this regard, reference may be made to Steinman et al. (International Publication WO 97/29182) who describe methods and compositions for isolation and maturation of dendritic cells.

Other methods for isolation, expansion and/or maturation of dendritic cells for the purpose of the present invention are described for example by Takamizawa et al (1997, J Immunol, 158 (5): 2134-2142), Thomas and Lipsky (1994, J Immunol, 153 (9): 4016-4028), O'Doherty et al. (1994, Immunology, 82 (3): 487-93), Fearnley et al. (1997, Blood, 89 (10): 3708-3716), Weissman et al. (1995, Proc Natl Acad Sci USA, 92 (3): 826-830), Freudenthal and Steinman (1990, Proc Natl Acad Sci USA, 87 (19): 7698-7702), Romani et al. (1996, J Immunol Methods, 196 (2): 137-151), Reddy et al. (1997, Blood, 90 (9): 3640-3646), Thurnher et al. (1997, Exp Hematol, 25 (3): 232-237), Caux et al. (1996, J Exp Med, 184 (2): 695-706; 1996, Blood, 87 (6): 2376-85), Luft et al. (1998, Exp Hematol, 26 (6): 489-500; 1998, J Immunol, 161 (4): 1947-1953), Cella et al. (1999, J Exp Med, 189 (5): 821-829; 1997, Nature, 388 (644): 782-787; 1996, J Exp Med, 184 (2): 747-572), Ahonen et al. (1999, Cell Immunol, 197(1): 62-72) and Piemonti et al. (1999, J Immunol, 162 (11): 6473-6481).

Alternatively, transformed or immortalised dendritic cell lines may be produced using oncogenes such as v-myc as for example described by Paglia et al. (1993, J Exp Med, 178 (6): 1893-1901).

1.2 Antigen Priming of Antigen-Presenting Cells

The amount of antigen to be placed in contact with antigen-presenting cells, which are preferably dendritic cells, can be determined empirically by persons of skill in the art. Antigen-presenting cells (which are preferably dendritic cells) are incubated with the antigen for 1-2 hr at 37° C. For most antigens, 10 μg/mL to 1-10 million dendritic cells is suitable for priming the dendritic cells. In a preferred embodiment, immature dendritic cells are utilised for the antigen internalisation.

The antigen should be exposed to the antigen-presenting cells for a period of time sufficient for the antigen-presenting cells to internalise the antigen. The time necessary for the cells to internalise and present the processed antigen may be determined using pulse-chase protocols in which exposure to antigen is followed by a washout period. Once the minimum time necessary for cells to express processed antigen on their surface is determined, a protocol may be used to prepare cells and antigen for eliciting immunogenic responses. Those of skill in the art will recognise in this regard that the length of time necessary for an antigen-presenting cell to phagocytose or internalise an antigen may vary depending on the antigen used. Efficiency of priming of the antigen-presenting cells can be determined by assaying T cell cytotoxic activity in vitro or using antigen-presenting cells as targets of CTLs. Other methods known to practitioners in the art, which can detect the presence of antigen on the surface of antigen-presenting cells after exposure to antigen, are also contemplated by the presented invention.

In another embodiment, a nucleic acid construct is introduced into an antigen-presenting cell, which construct comprises a polynucleotide, which encodes the aberrant polypeptide product and which is operably connected to a transcriptional control element that is functional in the antigen-presenting cell. Alternatively, a polynucleotide, which encodes the aberrant polypeptide product and which can be translated directly in the antigen-presenting cell (e.g., mRNA) can be introduced into the antigen-presenting cell, as for example described by Heiser et al. (2002, J. Clin. Invest. 109(3): 409-417). Delivery of the construct or polynucleotide to the antigen-presenting cell may be achieved using any suitable technique known to those of skill in the art including, for example, microprojectile bombardment, liposome mediated transfection (e.g., lipofectin or lipofectamine), electroporation, calcium phosphate or DEAE-dextran-mediated transfection. A discussion of suitable delivery methods may be found in Chapter 9 of CURRENT PROTOCOLS IN MOLECULAR BIOLOGY (Eds. Ausubel et al.; John Wiley & Sons Inc., 1997 Edition). Once transfected, the antigen-presenting cell is cultured for a time and under conditions for expression of the aberrant polynucleotide product and for processing of this product for presentation to, and modulation of, T cells.

In preferred embodiments where the antigen-presenting cells are dendritic cells, such cells have the capacity to efficiently present a processed form of the aberrant polypeptide product in the form of peptides on both MHC class I and class II molecules. Antigens are acquired by dendritic cells through the exogenous pathway by phagocytosis and, as a result, efficiently charge MHC class II molecules. Accordingly, both CD4⁺ T helper lymphocytes and CTL may be activated by dendritic cells presenting modified and optionally unmodified antigen in the context of MHC class II. These lymphocytes can provide critical sources of help, both for generating active CD8⁺ CTL and can in some circumstances be primed as CD4⁺ CTL with specificity for the target antigen during the acute response to antigen, and for generating the memory that is required for long term resistance and vaccination. Further, antigen uptake and presentation by dendritic cells, allows these cells to tailor the peptides that are appropriate for an individuals MHC products, and increases the number of specialised stimulatory antigen-presenting cells. Moreover, dendritic cells can be charged with multiple antigens on multiple MHCs to yield polyclonal or oligoclonal stimulation of T cells. Thus, by using the antigens of the present invention to charge MHC class I and class II molecules, efficient T cell modulation in situ can be achieved.

1.3 Methods for Assessing Immunomodulation

The effectiveness of the immunization may be assessed using any suitable technique. For example, CTL lysis assays may be employed using stimulated splenocytes or peripheral blood mononuclear cells (PBMC) on peptide coated or recombinant virus infected cells using ⁵¹Cr labeled target cells. Such assays can be performed using for example primate, mouse or human cells (Allen et al., 2000, J. Immunol. 164(9): 4968-4978 also Woodberry et al., infra). Alternatively, the efficacy of the immunization may be monitored using one or more techniques including, but not limited to, HLA class I tetramer staining—of both fresh and stimulated PBMCs (see for example Allen et al., supra), proliferation assays (Allen et al., supra), ELISPOT assays and intracellular cytokine staining (Allen et al., supra), ELISA Assays—for linear B cell responses; and Western blots of cell sample expressing the synthetic polynucleotides. Particularly relevant will be the cytokine profile of T cells activated by antigen, and more particularly the production and secretion of IFN γ, IL-2, IL4, IL5, IL-10, TGFβ and TNF α.

The cytotoxic activity of T lymphocytes, and in particular the ability of cytotoxic T lymphocytes to be induced by antigen-presenting cells, may be assessed by any suitable technique known to those of skill in the art. For example, a sample comprising T lymphocytes to be assayed for cytotoxic activity is obtained and the T lymphocytes are then exposed to antigen-primed antigen-presenting cells, which have been caused to present antigen. After an appropriate period of time, which may be determined by assessing the cytotoxic activity of a control population of T lymphocytes which are known to be capable of being induced to become cytotoxic cells, the T lymphocytes to be assessed are tested for cytotoxic activity in a standard cytotoxic assay. Such assays may include, but are not limited to, the chromium release CTL assay known in the art.

5. Compositions and Uses Therefore

The modulatory agents described respectively in Section 3 and the antigen-primed antigen-presenting cells described in Section 4 (therapeutic agents) can be used as active ingredients in pharmaceutical compositions for treatment or prophylaxis of prostate cancer. The invention, therefore, also extends to a method for treating or preventing prostate cancer, comprising administering to a patient in need of such treatment an effective amount of a therapeutic agent as broadly described above.

A pharmaceutical composition according to the invention is administered to a patient, preferably prior to such symptomatic state associated with prostate cancer. The therapeutic agent present in the composition is provided for a time and in a quantity sufficient to treat that patient. Suitably, the pharmaceutical composition comprises a pharmaceutically acceptable carrier.

Depending on the specific conditions being treated, therapeutic agents may be formulated and administered systemically or locally. Techniques for formulation and administration may be found in “Remnington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the cancer or tumour. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate tissue levels of a polypeptide, fragment, variant or derivative of the invention, and progression of the disorder. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement of K2 10A or PSA RP2 activity). Such information can be used to more accurately determine useful doses in humans.

Toxicity and therapeutic efficacy of such therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain K2 10A- or PSA RP2-inhibitory effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably a kidney tissue, a stomach tissue or a rectal tissue, often in a depot or sustained release formulation. Furthermore, one may administer the drug in a targeted drug delivery system, for example, in a liposome coated with tissue-specific antibody. The liposomes will be targeted to and taken up selectively by the tissue. In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

Tumour Samples

Up to 24 Prostate cancer and 28 benign prostatic hyperplasia (BPH) samples were obtained from men who underwent either transurethral resection of the prostate (TURP) or radical prostatectomy at the Royal Brisbane Hospital (RBH), Brisbane. Each sample was histologically analysed to confirm pathology at the RBH. Ethics approval was obtained from the respective institutional Ethics Committees and informed consent was obtained from all patients.

### Example 2

RT-PCR for Determining PSA and KLK2 mRNA Species in Prostate Cancer and BPH Tissues

Total RNA was extracted from tissues either at QUT or the RBH using TR1-Reagent (Sigma) following the manufacturer's instructions. For complementary DNA (cDNA) synthesis, 1-2 μg of total RNA was reverse transcribed using Superscript II (Invitrogen). Primers, specific for PSA wild-type, PSA 525, PSA (Schulz), KLK2 wild type, KLK2 10A and 12-microglobulin as well as primer common to PSA RP2 transcripts 1 and 2 (Table 1) were used in 20 μL reactions containing: 10× buffer containing 1.5 mmol/L Mg⁺ (Roche), 10 mmol/L deoxynucleotide triphosphates (dATP, dGTP, dCTP, dTTP), 100 ng/μL primers, 2.5 units of Taq (Roche) and 0.5 μL of cDNA. The PCR cycling conditions were 94° C. for 4 min, followed by 35 cycles of denaturation at 94° C. for 1 min, primer annealing at 56-60° C. for 1 min (specific to each transcript primer combination), and extension at 72° C. for 1 min, with a final extension of 8 min. All PCR products were electrophoresed on 1.5% TAE-agarose gels.

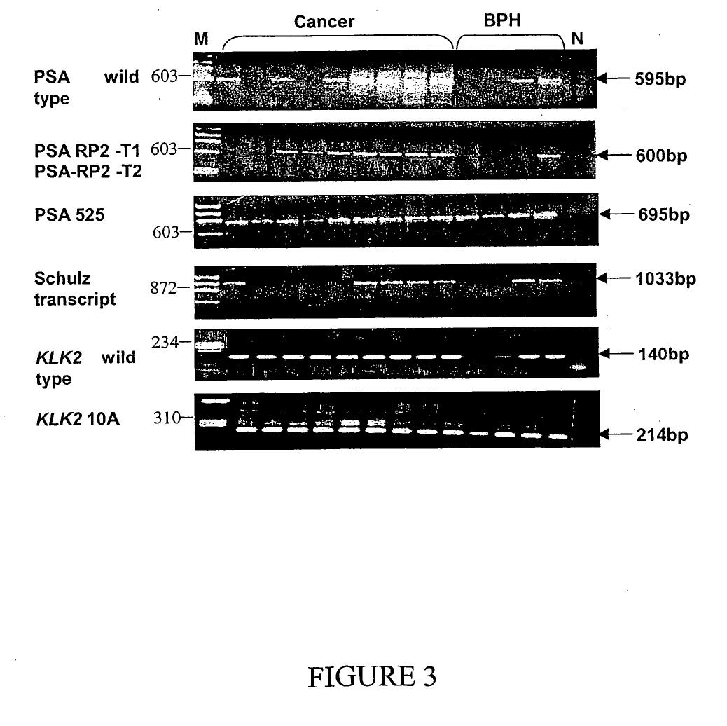

These results show that although the PSA and KLK2 wild type and variant transcripts were present in both BPH and cancer samples, there was a trend towards greater expression in the cancer samples (FIG. 3). Thus, real-time PCR of a larger number of samples was performed.

A number of PCR products were sequenced to determine primer specificity using the ABI PRISM Dye Terminator Cycle Sequencing Ready Reaction Kit (Perkin Elmer) at the DNA sequencing facility, University of Queensland, Brisbane.

### Example 3

Real-Time PCR Analysis of PSA and KLK2 Expression in Prostate Cancer and BPH

Standard PCR products for each transcript assayed (PSA wild-type, PSA RP2 transcript 2, PSA 525, PSA (Schulz), KLK2 wild type, KLK2 10A and O₂-microglobulin) were made by amplifying transcript-specific products as described above, purifying them using a gel extraction kit (Qiagen) and calculating the DNA copy number before serially diluting them for use in a standard curve (10³-10¹⁰ copies/μL). The Idaho Technology LC32 was used to determine the transcript copy number for each individual tissue sample. 10 ΔL PCR reactions were set up with 10×PCR buffer containing 30 mM MgCl₂ and 1 mg/mL BSA (Idaho Technology), 0.2 mM dNTPs, 100 ng/mL forward and reverse primers (Table 2), 0.5× SYBR Green I (Molecular Probes), 0.25 units of Platinum Taq (Invitrogen) and 1 μL of template (either tissue cDNA or standard with a known copy number). The reactions were transferred to LC capillary tubes (Idaho Technology) and reactions were cycled as follows: 94° C. for 2 min, 50 cycles of denaturation at 94° C. for 1 sec, annealing at 55-65° C. for 2 sec (depending on primer combination), and extension for 20 sec with fluorescence readings taken at 2° C. below the resultant melting temperature (86-90° C.). Continuous fluorescence readings with temperature transitions of 0.2° C./sec between 72-94° C. resulted in melting curve analysis of each transcript.

For each mRNA species assayed, a transcript-specific standard curve was generated by the LC32 software (10³-10¹⁰ copies/μL). From this standard curve, DNA copy numbers for each individual tissue cDNA could be calculated. Each assay was completed twice, in duplicate, and copy numbers were normalised to 62-microglobulin levels collected and averaged for all tissues analysed using the LC32. Normalised values for each sample population were averaged and statistical analysis was done to determine whether the two sample groups are likely to have come from the same two underlying populations by using the student's t-test.

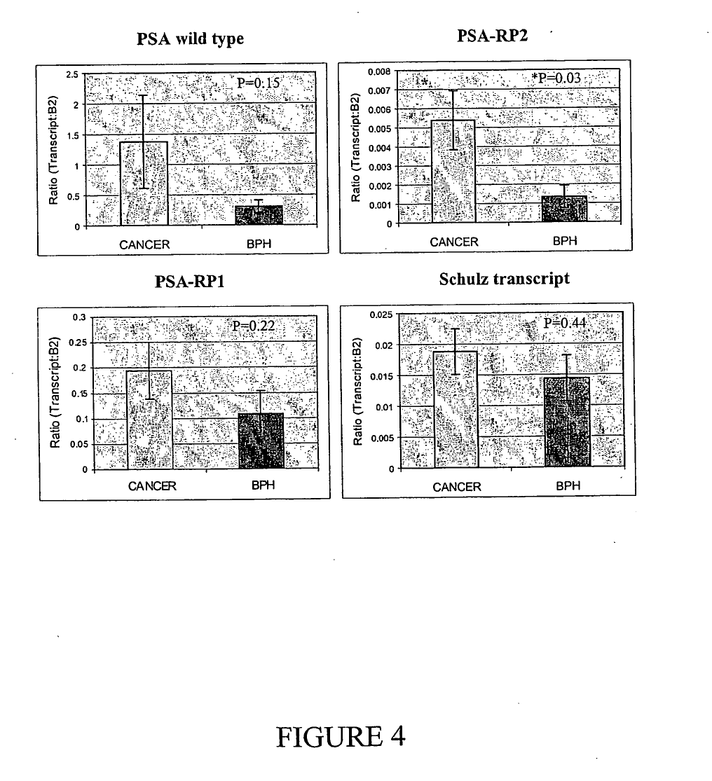

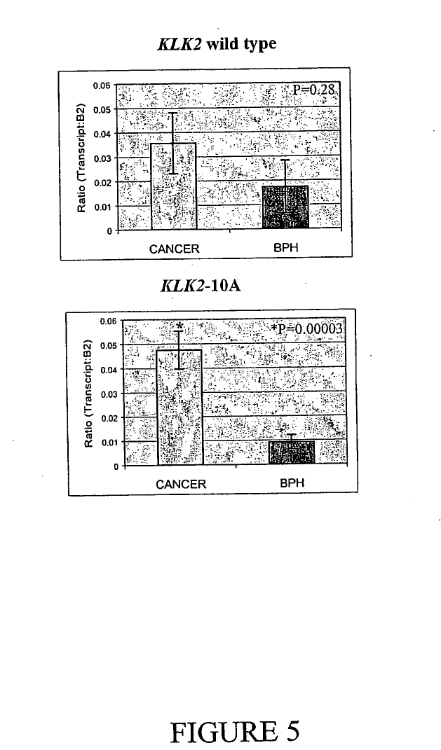

Analysis of real-time PCR normalised values using a Student's t-test, shows that there is a significant difference between the two groups for PSA RP2 transcript 2 and KLK2 10A, but not wild-type PSA, PSA 525, PSA Schulz or wild-type KLK2 (FIGS. 4 and 5, respectively). In both cases, expression of the variant forms are highly expressed in the cancer population, but not the benign disease, BPH. Therefore, if found to be expressed in the serum of patients with prostate cancer and BPH, these variant forms may become useful as potential markers of prostate cancer.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1
Primer sequences for qualitative RT-PCR

| | | | |
|---|---|---|---|
| | | Annealing | Product |
| PCR Product | Primer sequence | temp. (° C.) | size (bp) |

| | | | | |
|---|---|---|---|---|
| PSA wilt-type | For 5′-ATCGAATTCGCACCCGGAGAGCTGTGT-3′ | [SEQ ID NO: 16] | 60 | 595 |
| | Rev 5′-CTGAGGGTGAACTTGCGCACAC-3′ | [SEQ ID NO: 17] | | |
| PSA RP2 | For 5′-ATCGAATTCGCACCCGGAGAGCTGTGT-3′ | [SEQ ID NO: 18] | 60 | 600 |
| transcripts 1 and 2 | Rev 5′-CAAGGCAAAAAACACTGTTGT-3′ | [SEQ ID NO: 19] | | |
| PSA 525 | For 5′-GGTATTTCAGGTCAGCCACAG-3′ | [SEQ ID NO: 20] | 63 | 695 |
| | Rev 5′-TAAAAGCAGGGAGAGAGTGAG-3′ | [SEQ ID NO: 21] | | |
| PSA (Schulz) | For 5′-CTGGGCGCTGTCTTGTGTCTC-3′ | [SEQ ID NO: 22] | 64 | 1033 |
| | Rev 5′-ATGTCTAGAGTTGATAGGGGTGCTCAG-3′ | [SEQ ID NO: 23] | | |
| KLK2 wild type | For 5′-GCCTAAAGAAGAATAGCCAGGT-3′ | [SEQ ID NO: 24] | 58 | 140 |
| | Rev 5′-TCTAAGGCTTTGATGCTTCAG-3′ | [SEQ ID NO: 25] | | |
| KLK2 10A | For 5′-CACTTGTGGGGTGAGTCATCC-3′ | [SEQ ID NO: 26] | 60 | 214 |
| | Rev 5′-ATGTCTAGATGGGACAGGGGCACTCAG-3′ | [SEQ ID NO: 27] | | |
| β2-microgloblin | For 5′-TGAATTGCTATGTGTCTGGGT-3′ | [SEQ ID NO: 28] | 56 | 238 |
| | Rev 5′-CCTCCATGATGCTGCTTACAT-3′ | [SEQ ID NO: 29] | | |

TABLE 2
Primer sequences for quantitative Real Time PCR

| | | | |
|---|---|---|---|
| | | | Melting |
| | | Annealing | temp. |
| PCR Product | Primer sequence | temp. (° C.) | (° C.) |

| | | | | |
|---|---|---|---|---|
| PSA wild-type | For 5′-GCATCAGGAACAAAAGCGTGA-3′ | [SEQ ID NO: 30] | 58 | 84 |
| | Rev 5′-CCTGAGGAATCGATTCTTCAG-3′ | [SEQ ID NO: 31] | | |
| PSA RP2 | For 5′-GAGTGTACGCCTGGGCCAGAT-3′ | [SEQ ID NO: 32] | 65 | 90 |
| transcript 2 | Rev 5′-CTGAGGGTGAACTTGCGCACAC-3′ | [SEQ ID NO: 33] | | |
| PSA 525 | For 5′-CTCACTCTCTCCCTGCTTTTA-3′ | [SEQ ID NO: 34] | 60 | 88 |
| | Rev 5′-ATGTCTAGAGTTGATAGGGGTGCTCAG-3′ | [SEQ ID NO: 35] | | |
| PSA (Schulz) | For 5′-CTGGGCGCTGTCTTGTGTCTC-3′ | [SEQ ID NO: 36] | 65 | 88 |
| | Rev 5′-CAACGAGAGGCCACAAGCACC-3′ | [SEQ ID NO: 37] | | |
| KLK2 wild type | For 5′-GCCTAAAGAAGAATAGCCAGGT-3′ | [SEQ ID NO: 38] | 58 | 88 |
| | Rev 5′-TCTAAGGCTTTGATGCTTCAG-3′ | [SEQ ID NO: 39] | | |
| KLK2 10A | For 5′-CACTTGTGGGGTGAGTCATCC-3′ | [SEQ ID NO: 40] | 60 | 88 |
| | Rev 5′-ATGTCTAGATGGGACAGGGGCACTCAG-3′ | [SEQ ID NO: 41] | | |
| B2-microgloblin | For 5′-TGAATTGCTATGTGTCTGGGT-3′ | [SEQ ID NO: 42] | 55 | 84 |
| | Rev 5′-CCTCCATGATGCTGCTTACAT-3′ | [SEQ ID NO: 43] | | |

1-57. (canceled)

58. A method for detecting the presence or diagnosing the risk of prostate cancer in a patient, comprising detecting in a biological sample obtained from the patient a level or functional activity of an aberrant expression product of a gene selected from the group consisting of PSA and KLK2, which level or functional activity correlates with the presence or risk of prostate cancer, wherein the aberrant expression product is selected from the group consisting of PSA RP2 transcript 1, PSA RP2 transcript 2, KLK2 10A transcript and a polypeptide encoded by any one of these transcripts.

59. The method of claim 1, wherein the aberrant expression product is a transcript comprising the sequence set forth in SEQ ID NO: 7.

60. The method of claim 1, wherein the aberrant expression product is a transcript comprising the sequence set forth in SEQ ID NO: 1.

61. The method of claim 1, wherein the aberrant expression product is a transcript comprising the sequence set forth in SEQ ID NO: 3.

62. The method of claim 1, wherein the aberrant expression product is a transcript comprising the sequence set forth in SEQ ID NO: 5.

63. The method of claim 1, wherein the aberrant expression product is a polypeptide comprising the sequence set forth in SEQ ID NO: 2.

64. The method of claim 1, wherein the aberrant expression product is a polypeptide comprising the sequence set forth in SEQ ID NO: 4.

65. The method of claim 1, wherein the aberrant expression product is a polypeptide comprising the sequence set forth in SEQ ID NO: 6.

66. The method of claim 1, wherein the aberrant expression product is a transcript comprising the sequence set forth in SEQ ID NO: 9.

67. The method of claim 1, wherein the aberrant expression product is a transcript comprising the sequence set forth in SEQ ID NO: 11.

68. The method of claim 1, wherein the aberrant expression product is a transcript comprising the sequence set forth in SEQ ID NO: 13.

69. The method of claim 1, wherein the aberrant expression product is a polypeptide comprising the sequence set forth in SEQ ID NO: 10.

70. The method of claim 1, wherein the aberrant expression product is a polypeptide comprising the sequence set forth in SEQ II) NO: 12.

71. The method of claim 1, wherein the aberrant expression product is a polypeptide comprising the sequence set forth in SEQ ID NO: 14.

72. The method of claim 1, wherein the correlation with the presence or risk of prostate cancer is made when the level or functional activity of the aberrant expression product in the biological sample is at least. 10% higher than a reference level or functional activity of the expression product that correlates with the presence or risk of benign prostatic hyperplasia.

73. The method of claim 1, further comprising quantifying an aberrant polypeptide product of the gene.

74. The method of claim 1, further comprising quantifying an aberrant polypeptide product of the gene, wherein the aberrant polypeptide product is quantified by:
contacting the biological sample with an antigen-binding molecule that is immunointeractive with the aberrant polypeptide product;
measuring the concentration of a complex comprising the aberrant polypeptide product and the antigen binding molecule in the contacted sample; and
relating the measured complex concentration to the concentration of the aberrant polypeptide product in the sample.

75. The method of claim 17, wherein the concentration of the aberrant polypeptide product in the biological sample is compared to a reference level of the aberrant polypeptide product which correlates with the presence or risk of benign prostatic hyperplasia.

76. The method of claim 1, further comprising measuring the level of an aberrant transcript product of the gene in the biological sample.

77. The method of claim 19, wherein the level of the transcript in the biological sample is compared to a reference level of the transcript which correlates with the presence or risk of benign prostatic hyperplasia.

78. The method of claim 19, wherein the level of the transcript is measured using a probe that comprises a nucleotide sequence which corresponds or is complementary to at least a portion of the aberrant transcript.

79. The method of claim 19, wherein the level of an aberrant transcript is quantified using a nucleic acid amplification technique that quantifies the aberrant transcript in real-time.

80. The method of claim 1, comprising indirectly analysing the level of an aberrant polypeptide product of the gene.

81. The method of claim 1, comprising indirectly analysing the level of an aberrant polypeptide product by qualitatively or quantitatively determining in the biological sample the level of an antigen-binding molecule that is immuno-interactive with the aberrant polypeptide product.

82. The method of claim 24, comprising:
contacting the biological sample with an antigen corresponding to at least a portion of the aberrant polypeptide product;
measuring the concentration of a complex comprising the antigen and an antigen-binding molecule in the contacted sample; and
relating the measured complex concentration to the concentration of antigen-binding molecule in the sample to thereby determine the amount or level of the aberrant polypeptide product in the sample.

83. The method of claim 1, wherein the prostate cancer is metastatic prostate cancer.

84. The method of claim 26, wherein the metastatic prostate cancer is associated with metastasis to a bone or lymph node of the patient.

85. The method of claim 1, wherein the expression product is present intracellularly.

86. The method of claim 1, wherein the expression product is present in soluble form.

87. The method of claim 29, wherein, the biological sample comprises a biological fluid selected from seminal fluid, whole blood, serum or lymphatic fluid.

88. The method of claim 1, wherein the prostate cancer is an organ-confined prostate cancer.

89. A method for detecting the presence or diagnosing the risk of prostate cancer in a patient, comprising detecting in a biological sample obtained from the patient a level or functional activity of an aberrant expression product of a gene selected from the group consisting of PSA and KLK2, which level or functional activity correlates with the presence or risk of prostate cancer.

90. A method for detecting the presence or diagnosing the risk of prostate cancer in a patient, comprising detecting in a biological sample obtained from the patient a level or functional activity of an aberrant expression product of a gene selected from the group consisting of PSA and KLK2, which level or functional activity is higher than a reference level or functional activity of the expression product which correlates with the presence or risk of benign prostatic hyperplasia.

91. A method for detecting the presence or diagnosing the risk of prostate cancer in a patient, comprising detecting in a biological sample obtained from the patient a level or functional activity of a transcript selected from the group consisting of PSA RP2 transcript 1, PSA RP2 transcript 2 and KLK2 10A, which level or functional activity correlates with the presence or risk of prostate cancer.

92. A method for detecting the presence or diagnosing the risk of prostate cancer in a patient, comprising detecting in a biological sample obtained from the patient a level or functional activity of a transcript selected from the group consisting of PSA RP2 transcript 2 and KLK2 10A, which level or functional activity correlates with the presence or risk of prostate cancer.

93. A method for detecting the presence or diagnosing the risk of metastatic prostate cancer in a patient, comprising detecting in a biological sample obtained from the patient a level or functional activity of an aberrant expression product of a gene selected from the group consisting of PSA and KLK2, which level or functional activity correlates with the presence or risk of prostate cancer, wherein the biological sample comprises a fluid or tissue other than prostate tissue.

94. A method for detecting the presence or diagnosing the risk of organ-confined prostate cancer in a patient, comprising detecting in a biological sample obtained from the patient the absence of a level or functional activity of an aberrant expression product of a gene selected from the group consisting of PSA and KLK2, which correlates with the presence or risk of prostate cancer, wherein the biological sample comprises a fluid or tissue other than prostate tissue.

95. A method for the treatment and/or prophylaxis of a prostate cancer, comprising administering to a patient in need thereof an effective amount of an agent selected from an antisense oligonucleotide, a ribozyme or an RNAi-mediating molecule that binds to, or otherwise eracts specifically with, an aberrant transcript of a gene selected from level or functional activity PSA and KLK2, whose level or functional activity correlates with the presence or risk of prostate cancer, wherein the agent is optionally formulated with a pharmaceutically acceptable carrier.

96. A method for the treatment and/or prophylaxis of a prostate cancer, comprising administering to a patient in need thereof an effective amount of an antigen-binding molecule that is immuno-interactive with an aberrant polypeptide product of a gene selected from level or functional activity PSA and KLK2, whose level or functional activity correlates with the presence or risk of prostate cancer, wherein the antigen-binding molecule is optionally formulated with a pharmaceutically acceptable carrier.

97. A method for the treatment and/or prophylaxis of a prostate cancer, comprising administering to a patient in need thereof an effective amount of an antigen-binding molecule that is immuno-interactive with an aberrant polypeptide product of a gene selected from level or functional activity PSA and KLK2, whose level or functional activity correlates with the presence or risk of prostate cancer, wherein the antigen-binding molecule is optionally formulated with a pharmaceutically acceptable carrier.

98. A method for the treatment and/or prophylaxis of a prostate cancer, comprising administering to a patient in need thereof an effective amount of an antigen-presenting cell expressing a processed form of an aberrant polypeptide product of a gene selected from level or functional activity PSA and KLK2 for presentation to, and modulation of, T cells, which level or functional activity of the expression product correlates with the presence or risk of prostate cancer, wherein the antigen-presenting cell is optionally formulated with a pharmaceutically acceptable carrier.
